(19) United States
(12) Patent Application Publication
(10) Pub. No.: US 2026/0053498 A1
(43) Pub. Date: Feb. 26, 2026

(54) SUTURING DEVICE

(71) Applicant: LSI Solutions, Inc., Victor, NY (US)

(72) Inventor: Jude S. Sauer, MD, Pittsford, NY (US)

(21) Appl. No.: 19/307,104

(22) Filed: Aug. 22, 2025

Related U.S. Application Data
(60) Provisional application No. 63/685,915, filed on Aug. 22, 2024.

(51) Int. Cl. A61B 17/04

(52) U.S. Cl. CPC A61B 17/0482

(57) ABSTRACT

A suturing device includes an actuation lever having a displaceable actuation lever having a cam path is formed in a portion of the actuation lever. A cam follower of a follower plate is disposed in the cam path such that as the actuation lever is displaced from a first position to an intermediate position, the follower plate is displaced from a proximal position to a distal position thereby displacing a first needle coupled to the follower plate from a first position to a second position, and as the actuation lever is displaced from the intermediate position to a third position, the follower plate is displaced from the distal position to the proximal position, thereby displacing the first needle from the second position to the first position.

### CROSS-REFERENCE TO RELATED APPLICATIONS

This application claims the benefit of U.S. Provisional Patent Application No. 63/685,915, filed Aug. 22, 2024, the contents of which is incorporated by reference herein in its entirety.

### FIELD OF THE INVENTION

The claimed invention relates to surgical devices, and more specifically to a surgical suturing device.

### BACKGROUND OF THE INVENTION

Stay sutures are temporary surgical sutures that are placed to hold and manipulate tissue during surgical procedures. The stay sutures provide controlled tensioning and manipulation of tissue for improved exposure to the surgical site. Stay sutures are typically placed using a surgical needle that is held and controlled by surgical graspers or a laparoscopic needle driver. However, there are several potential issues associated with each of these placement techniques. For example, graspers may not provide the same secure grip on a needle as needle holders, increasing the risk of the needle slipping or rotating during suturing, which can lead to inaccurate suture placement and potentially damage surrounding tissues. Furthermore, graspers may lack the necessary features for delicate maneuvers that require precise movements and fine control over the needle. With respect to needle drivers, inadequate pressure from the needle driver's jaws can make it difficult to properly grasp and drive the needle through tissue, resulting in poor suture placement and potentially hindering wound healing. In addition, applying excessive pressure to the needle with the driver can deform or bend the needle, compromising its ability to pass smoothly through the tissue and potentially leading to inaccurate suturing. If the jaws of the driver are worn or damaged, the needle may slip during suturing, increasing the risk of accidental needle sticks to the surgeon or the surgical team. Moreover, using an ill-fitting or poorly designed needle driver can cause discomfort and fatigue, particularly during lengthy procedures, affecting the surgeon's dexterity, focus, and overall suturing performance. Accordingly, there is a need for a device that simplifies the placement of sutures, such as stay sutures, while achieving consistent suture placement that reduces the risk of unintended tissue contact with the placement needle.

### BRIEF SUMMARY OF THE INVENTION

An embodiment of a suturing device may include a housing having one or more surfaces defining an interior portion. The embodiment of the suturing device may also include an actuation lever coupled to a first portion of the housing, the actuation lever having a grip portion that is configured to be engaged by a user, wherein a slot defining a cam path is formed in a first portion of the actuation lever, the actuation lever configured to be displaceable from an unactuated first lever position to an intermediate second lever position to a fully actuated third lever position by displacing the actuation lever in a single direction. The embodiment of the suturing device may further include a follower plate that includes a cam follower that is disposed within the slot defining the cam path of the actuation lever such that (a) as the actuation lever is displaced from the unactuated first lever position to the intermediate second lever position, the follower plate is displaced from an unactuated proximal position to an actuated distal position, and (b) as the actuation lever is displaced from the intermediate second lever position to the fully actuated third lever position, the follower plate is displaced from the actuated distal position to the unactuated proximal position. The embodiment of the suturing device may additionally include a first needle that extends from a proximal end to a distal end along a first needle axis, with a first needle tip being disposed at the distal end of the first needle, wherein the proximal end of the first needle is coupled to a first portion of the follower plate such that (a) as the follower plate is displaced from the unactuated proximal position to the actuated distal position, the first needle is displaced from a first needle position to a second needle position, and such that (b) as the follower plate is displaced from the actuated distal position to the unactuated proximal position to the actuated distal position, the first needle is displaced from the second needle position to the first needle position.

### BRIEF DESCRIPTION OF THE DRAWINGS

FIGS. 1A to 1G are various views of an embodiment of a suturing device;

FIG. 2A is a view of the embodiment of the suturing device with a portion of a housing portion removed for clarity, with an actuation lever in an unactuated first lever position;

FIG. 2B is a view of the embodiment of the suturing device with the portion of the housing portion removed for clarity, with the actuation lever in an intermediate second lever position;

FIG. 2C is a view of the embodiment of the suturing device with the portion of the housing portion removed for clarity, with the actuation lever in a fully actuated third lever position;

FIG. 3A is a view of a distal end assembly of the embodiment of the suturing device with one or more needles in an unactuated first needle position;

FIG. 3B is a view of the distal end assembly of the embodiment of the suturing device with the one or more needles displacing from the unactuated first needle position towards an engaged second needle position;

FIG. 3C is a view of the distal end assembly of the embodiment of the suturing device with the one or more needles in the engaged second needle position with the first and second needle tips engaging first and second ferrules, respectively;

FIG. 3D is a view of the distal end assembly of the embodiment of the suturing device with the one or more needles displacing from the engaged second needle position towards the unactuated first needle position with the first and second needle tips engaging first and second ferrules, respectively;

FIG. 3E is a view of the distal end assembly of the embodiment of the suturing device with the one or more needles in the engaged second needle position with the first and second needle tips engaging first and second ferrules, respectively;

FIG. 4A is a side view of the distal end assembly of the embodiment of the suturing device;

FIG. 4B is a front view of the distal end assembly of the embodiment of the suturing device;

FIG. 4C is a cross-sectional view of the shaft of the embodiment of the suturing device taken along section line 4C-4C of FIG. 1A;

FIG. 4D is a perspective view of the cross-section taken along section line 4C-4C of FIG. 1A;

FIG. 5 is a view of the embodiment of the suturing device with the portion of the housing portion removed for clarity, with the actuation lever in the unactuated first lever position, and with the follower plate disposed in an unactuated proximal position;

FIGS. 6A to 6D are various views of the actuation lever of the embodiment of the suturing device;

FIGS. 7A to 7C are various views of the follower plate of the embodiment of the suturing device; and

FIG. 8 is a detailed view of the embodiment of the suturing device with a portion of a housing portion removed for clarity illustrating the selective retention feature.

### DETAILED DESCRIPTION OF THE INVENTION

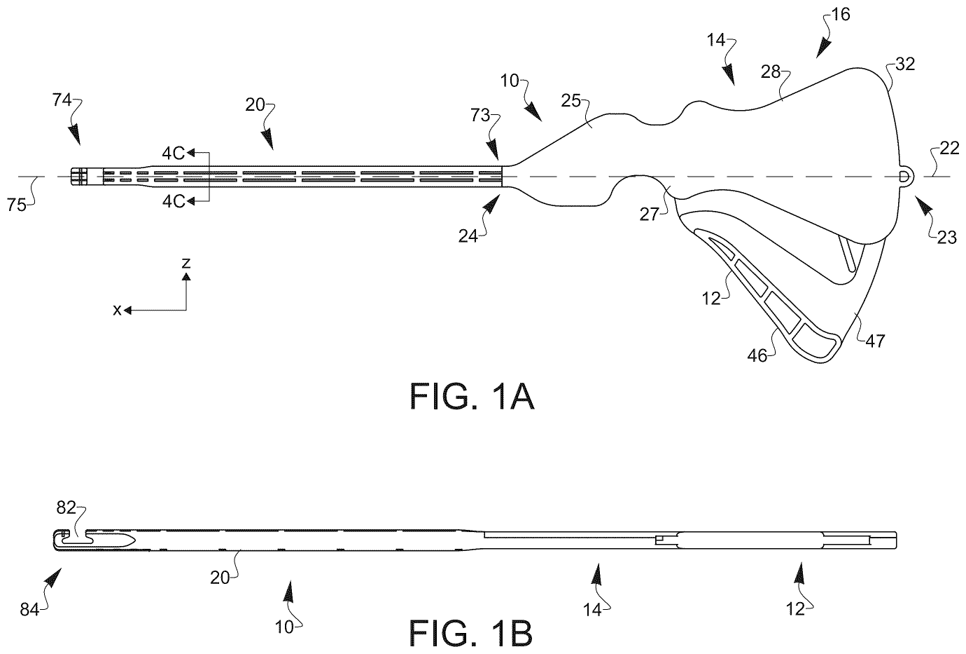

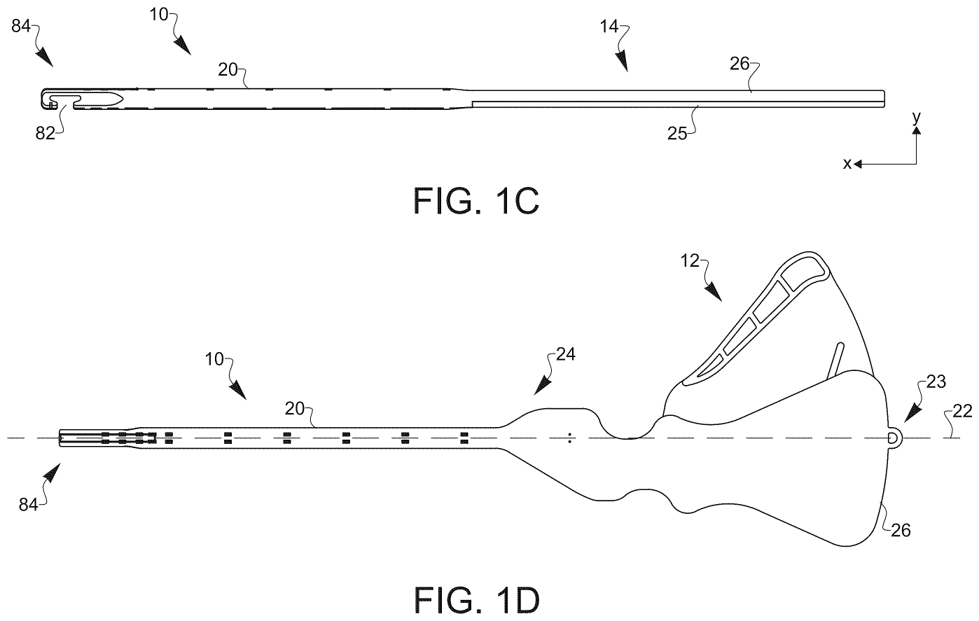

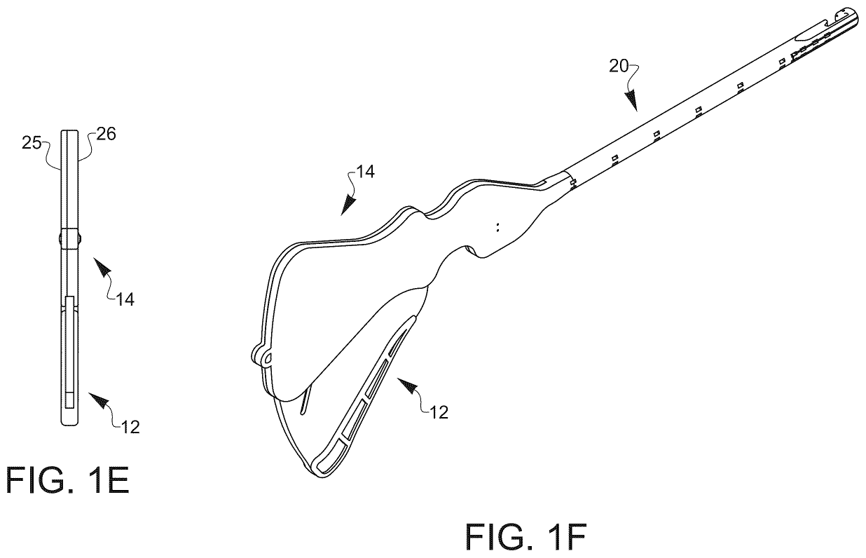

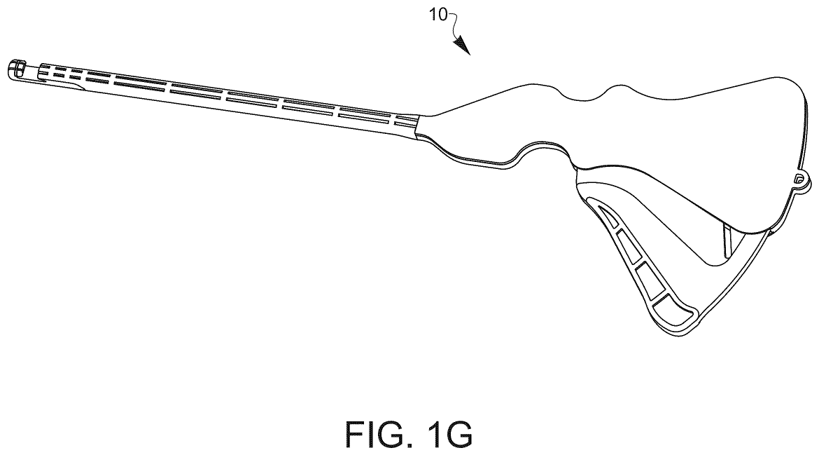

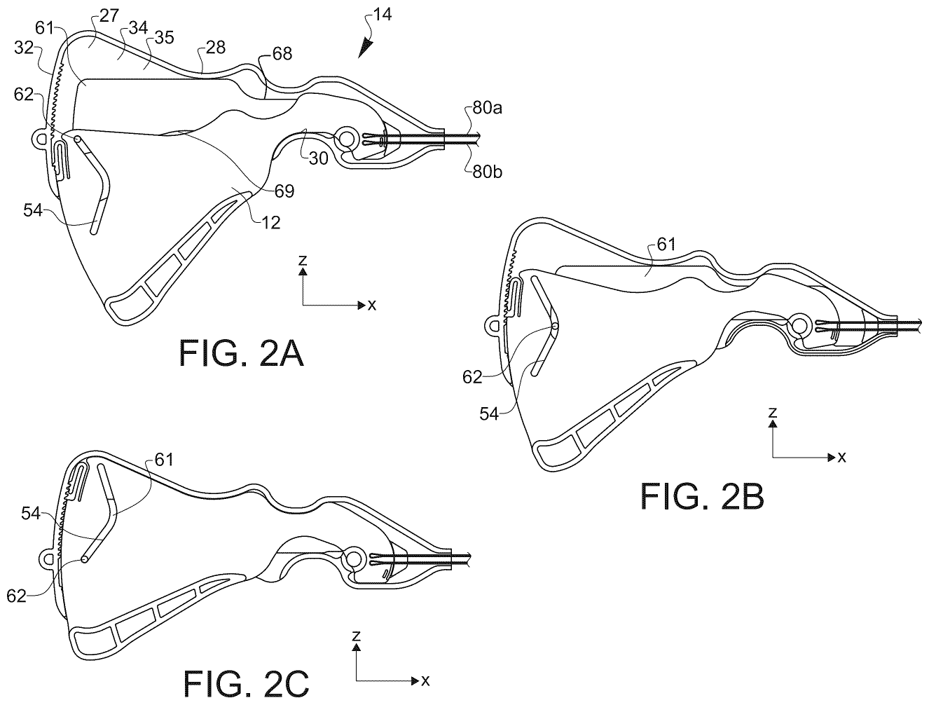

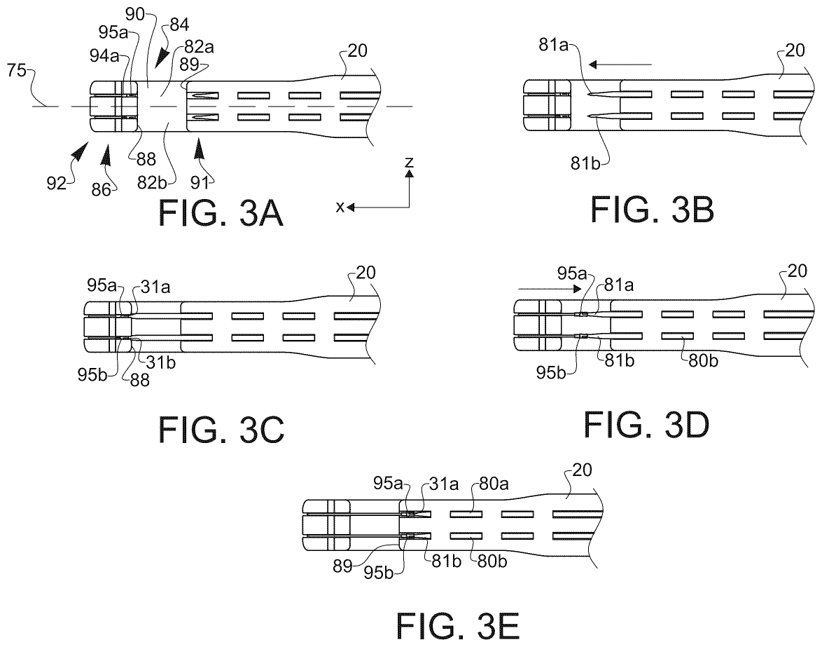

FIGS. 1A to 1G illustrate an embodiment of a suturing device 10 that includes a housing portion 14 having a grip portion 16 that is adapted to be grasped by a user to engage and displace an actuation lever 12 from an unactuated first lever position (illustrated in FIG. 2A) to an intermediate second lever position (illustrated in FIG. 2B) to a fully actuated third lever position (illustrated in FIG. 2C) by displacing the actuation lever 12 in a single direction. One or more elongated needles 80 are coupled to the actuation lever 12, and a portion of each of the one or more needles 80 extends through an interior portion of a shaft 20 that is coupled to the housing portion 14. When the actuation lever 12 is in the unactuated first lever position, each of the one or more needles 80 is in an unactuated first needle position (illustrated in FIG. 3A). As the actuation lever 12 is displaced from the unactuated first lever position towards the intermediate second lever position, each of the one or more needles 80 displaces from the unactuated first needle position towards an engaged second needle position (illustrated in FIG. 3B). When the actuation lever 12 is displaced to the intermediate second lever position, each of the one or more needles 80 is in the engaged second needle position (illustrated in FIG. 3C). As the actuation lever 12 is displaced from the intermediate second lever position towards the fully actuated third lever position, each of the one or more needles 80 displaces from the engaged second needle position towards the unactuated first needle position (illustrated in FIG. 3D). When the actuation lever 12 is displaced to the fully actuated third lever position, each of the one or more needles 80 is in the unactuated first needle position (illustrated in FIG. 3E). When the actuation lever 12 has been actuated from the unactuated first lever position to the fully actuated third lever position, a portion of the actuation lever 12 engages a portion of the housing portion 14 to retain the actuation lever in the fully actuated third lever position.

So configured, the suturing device 10 individually displaces two needles distally and proximally to precisely places two sutures at a desired treatment area with consistent needle pressure, all without the use of a needle driver or graspers. Further, both needles 80 are advanced distally and retracted proximally with the single upward squeeze of the actuation lever 12, simplifying the placement of the sutures. Further, the needles are advanced distally and retracted proximally within the shaft 20, and when the needles are retracted proximally, the needles are permanently retained in this proximal position to eliminate the danger of unintended damage or piercing by the needles 80. The suturing device 10 may also be pre-loaded so that a surgeon may quickly and easily position the tissue to be sutured in a tissue bite area 82 defined in a distal end assembly 84 at the distal end of the shaft 20. In addition, the suturing device 10 may be disposable after the single use for convenience.

With reference to FIG. 1A, the suturing device 10 may include the housing portion 14, which may be elongated and extend along a housing axis 22 from a proximal end 23 to a distal end 24, and the housing axis 22 may be parallel or substantially parallel to the X-axis of the reference coordinate system of FIG. 1A. The housing portion 14 may include a first lateral wall 27 having a first lateral external surface 25 that may extend from the proximal end 23 of the housing portion 14 to the distal end 24 of the housing portion 14, and the first lateral external surface 25 be planar and extend along a plane that may be parallel to the X-Z plane of the reference coordinate system of FIG. 1A. The housing portion 14 may also include a second lateral wall 29 having a second lateral external surface 26 that may extend from the proximal end 23 of the housing portion 14 to the distal end 24 of the housing portion 14, and the second lateral external surface 26 may be offset from the first lateral surface 25 along the Y-axis of the reference coordinate system of FIG. 1C. In some embodiments, the second lateral surface 26 may be planar and may be parallel to the first lateral surface 25 and may extend along a plane that may be parallel to the X-Z plane of the reference coordinate system of FIG. 1A.

The housing portion 14 may be further defined by a top edge portion 28 that may extend in a direction along the housing axis 22, and the top edge portion 28 may extend from a first end at or adjacent to the distal end 24 of the housing portion 14 to a second end at or adjacent to the proximal end 23 of the housing portion 14. In some embodiments, the top edge portion 28 may include one or more segments, and each of the two or more segments may be non-linear such that the top edge portion 28 is non-linear. The housing portion 14 may also be defined by a bottom edge portion 30 that may extend in a direction along the housing axis 22, and the bottom edge portion 30 may extend from a first end at or adjacent to the distal end 24 of the housing portion 14 to a second end at or adjacent to the proximal end 23 of the housing portion 14. In some embodiments, the bottom edge portion 30 may include two or more segments, and each of the two or more segments may be non-linear such that the bottom edge portion 30 is non-linear. In addition, the housing portion 14 may be defined by a proximal end edge 32 that may extend in a general direction normal to the housing axis 22, and the proximal end edge 32 may extend from the second end of the top edge portion 28 to the second end of the bottom edge portion 30. In some embodiments, the proximal end edge 32 may include two or more segments, or may include a single segment, and this segment may have the shape of an arc or a segment of a circle.

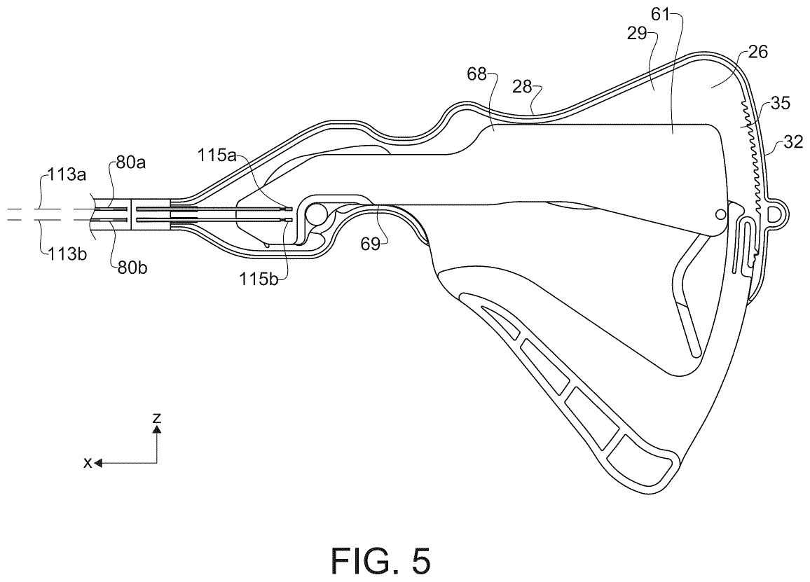

As illustrated in FIG. 2A (in which a portion of the housing portion 14 is omitted for clarity), the first lateral wall 27 may also include a first lateral internal surface 34. The first lateral internal surface 34 may be opposite to the first lateral external surface 25 of the first lateral wall 27, and the first lateral internal surface 34 may be planar and extend parallel to the first lateral external surface 25 of the first lateral wall 27. As illustrated in FIG. 5 (in which in which the first lateral wall 27 of the housing portion 14 is omitted for clarity), the second lateral wall 29 may also include a second lateral internal surface 36. The second lateral internal surface 36 may be opposite to the second lateral external surface 26 of the second lateral wall 29, and the second lateral internal surface 36 may be planar and extend parallel to the second lateral external surface 26 of the second lateral wall 29. The first lateral internal surface 34 and the second lateral internal surface 36 cooperate to at least partially define an interior portion 35 of the housing portion 14.

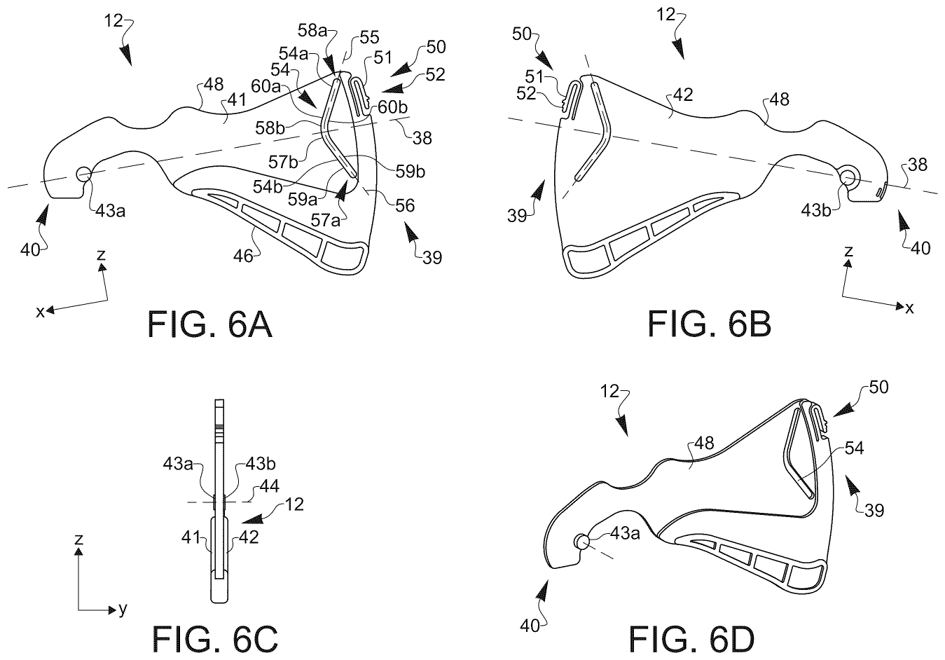

Referring to FIGS. 2A, 5, and 6A to 6D, the suturing device 10 may also include the actuator lever 12 that may be coupled to a portion of the housing portion 14. Turning to FIG. 6A, the actuator lever 12 may be elongated and extend along an actuator lever axis 38 from a proximal end 39 to a distal end 40, and the actuator lever axis 38 may be parallel or substantially parallel to the X-axis of the reference coordinate system of FIG. 6A. The actuator lever 12 may be planar and may include a first lateral surface 41 that may extend from the proximal end 39 of the actuator lever 12 to the distal end 40 of the actuator lever 12. The actuator lever 12 may also include a second lateral surface 42 that may extend from extend from the proximal end 39 of the actuator lever 12 to the distal end 40 of the actuator lever 12, and the second lateral surface 42 may be offset from the first lateral surface 41 along the Y-axis of the reference coordinate system of FIG. 6C.

Still referring to FIG. 6C, the actuation lever 12 may rotate about a rotational axis 44 from the unactuated first lever position (illustrated in FIG. 2A) through the intermediate second lever position (illustrated in FIG. 2B) to the fully actuated third lever position (illustrated in FIG. 2C). The rotational axis 44 may be disposed at or adjacent to the distal end 40 of the actuation lever 12, and the rotational axis 44 may be parallel to the Y-axis of the reference coordinate system of FIG. 6C. The rotational axis 44 may be disposed between the distal end 24 and the proximal end 23 of the housing portion 14, and the proximal end 39 of the actuation lever 12 may be disposed adjacent to the proximal end 23 of the housing portion 14.

In some embodiments, a first boss 43a may extend outwardly from the first lateral surface 41, and the first boss 43a may extend along a rotational axis that is coaxially-aligned with the rotational axis 44. In addition, a second boss 43b may extend outwardly from the second lateral surface 42, and the second boss 43b may extend along a rotational axis that is coaxially-aligned with the rotational axis 44. So configured, each of the first boss 43a and the second boss 43b may be received into corresponding features formed on or extending from the first lateral internal surface 34 of the housing portion 14 and the second lateral internal surface 36 of the housing portion 14, respectively, such the actuation lever 12 is configured to rotate about the rotational axis 44 from the unactuated first lever position to the fully actuated third lever position. With the first boss 43a and the second boss 43b disposed ass described, the first lateral surface 41 will be disposed opposing (and parallel to) the first lateral internal surface 34 of the housing portion 14, and the second lateral surface 42 will be disposed opposing (and parallel to) the second lateral internal surface 36 of the housing portion 14.

The actuation lever 12 may be further defined by a top edge portion 28 that may extend in a direction along the actuator lever axis 38, and the top edge portion 48 may extend from a first end at or adjacent to the distal end 40 of the actuation lever 12 to a second end at or adjacent to the proximal end 39 of the actuation lever 12. In some embodiments, the top edge portion 48 may include one or more segments, and each of the two or more segments may be non-linear such that the top edge portion 48 is non-linear. The top edge portion 48 of the actuation lever 12 may have a shape that is similar or identical to the top edge portion 28 of the housing portion 14, and the top edge portion 48 of the actuation lever 12 may be adjacent to the top edge portion 28 of the housing portion 14 when the actuation lever 12 is in the fully actuated third lever position. The top edge portion 28 may be disposed within the interior portion 35 of the housing portion 14 when the actuation lever 12 is rotated about the rotational axis 44 from the unactuated first lever position to the fully actuated third lever position.

The actuation lever 12 may also be defined by a bottom edge portion 46 that may extend in a general direction along the actuator lever axis 38, and the bottom edge portion 46 may extend from a first end at or adjacent to the distal end 40 of the actuation lever 12 to a second end at or adjacent to the proximal end 39 of the actuation lever 12. In some embodiments, the bottom edge portion 46 may include two or more segments, and each of the two or more segments may be non-linear such that the bottom edge portion 40 is non-linear. The bottom edge portion 46 may be disposed exterior to the interior portion 35 of the housing portion 14 when the actuation lever 12 is rotated about the rotational axis 44 from the unactuated first lever position to the fully actuated third lever position. The bottom edge portion 46 is configured such that when the actuation lever 12 is in the fully actuated third lever position, a portion of the bottom edge portion 46 is offset from a corresponding portion of the bottom edge portion 30 of the housing portion 14 such that a portion of the actuation lever 12 extends beyond the bottom edge portion 30 of the housing portion 14, as illustrated in FIG. 2C. When the actuation lever 12 is in the unactuated first lever position of FIG. 1A, the portion of the bottom edge portion 46 and a portion of the top edge portion 28 of the housing portion 14 cooperate to form a gripping portion that a user may grasp with a single hand to squeeze the actuation lever 12 from the unactuated first lever position to the fully actuated third lever position.

In addition, the actuation lever 12 may be defined by a proximal end edge 47 that may extend in a general direction normal to the actuator lever axis 38, and the proximal end edge 47 may extend from the second end of the top edge portion 48 to the second end of the bottom edge portion 46. In some embodiments, the proximal end edge 47 may include two or more segments, or may include a single segment, and this segment may have the shape of an arc or a segment of a circle. In some embodiments, the proximal end edge 47 may have an identical or substantially identical shape as the proximal end edge 32 of the housing portion 14, such as the segment of circle having an identical or substantially identical radius.

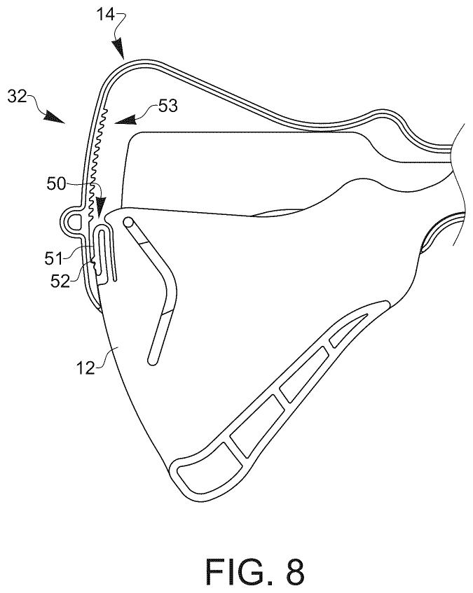

A selective retention feature 50, such as a cantilevered arm 51, may be disposed along the proximal end edge 47 at or adjacent to the second end of the top edge portion 48. In some embodiments, a first portion of the cantilevered arm 51 may extend in a direction along or substantially along or aligned with an adjacent portion of the proximal end edge 47, and the cantilevered arm 51 may have a lock feature 52 disposed at or adjacent to an end portion of the cantilevered arm 51. As illustrated in FIG. 8, the lock feature 52 may be positioned such that it contacts or slidingly engages each of a plurality of ratchet features 53 formed along an inner profile of the proximal end edge 32 of the housing portion 14 when the actuation lever 12 is displaced from the unactuated first lever position to the fully actuated third lever position. When the actuation lever 12 is to the fully actuated third lever position, the actuation lever 12 is prevented from being displaced towards the unactuated first lever position by the lock feature 52 that lockingly engages one or more of the plurality of ratchet features 53 formed along an inner profile of the proximal end edge 32 of the housing portion 14. In some embodiments, the lock feature 52 is a protrusion having a first contact surface that slides by each of the plurality of ratchet features 53 without lockingly engaging any of the plurality of ratchet features 53 when the actuation lever 12 is displaced from the unactuated first lever position to the fully actuated third lever position. However, the lock feature may have a second engagement surface (which may be opposite to the first contact surface), and the second engagement surface may be configured to lockingly engage surface of one or more of the plurality of ratchet features 53 when the actuation lever 12 is displaced towards the unactuated first lever position. Thus, when the actuation lever 12 is displaced from the unactuated first lever position to the fully actuated third lever position, the actuation lever 12 is retained in the fully actuated third lever position.

The actuation lever 12 may also include a cam path 54 that extends through one or more portions of the actuation lever 12. The cam path 54 may be an elongated slot or groove that extends along a portion of the actuation lever 12 and extends through a portion of the first lateral surface 41 to the second lateral surface 42. The cam path 54 may be configured to slidingly receive a cam follower 62 (as illustrated in FIG. 2A), which will be described in more detail in a following section.

The cam path 54 may include one or more segments, such as a first segment 54a and a second segment 54b. In some embodiments, the first segment 54a may be an elongated slot that extends from a first end 58a to a second end 58b along a first segment axis 55, and the second segment 54a may be an elongated slot that extends from a first end 57a to a second end 57b along a second segment axis 56. In some embodiments, each of the first segment axis 55 and the second segment axis 56 may be linear or substantially linear. However, all or one or more portions of one or both of the first segment axis 55 and the second segment axis 56 may be non-linear. In some embodiments, the cam path 54 may include more than two segments. The second end 58b of the first segment 54a may extend to the second end of the second segment 54b such that the first segment 54a and the second segment 54b form a continuous slot from the first end 58a of the first segment 54a to the first end 57a of the second segment 54b. A first length of the first segment 54a may extend from the first end 58a to the second end 58b, and a second length of the second segment 54b may extend from the first end 57a to the second end 57b. In some embodiments, the first length may be equal to or substantially equal to the second length. However, the first length may greater than or less than the second length. The first segment 54a may be at least partially defined by an opposing pair of lateral walls 59a, 59b and the second segment 54b may be at least partially defined by an opposing pair of lateral walls 60a, 60b, and the second end of the lateral wall 59a may intersect the second end of the lateral wall 60a, and the second end of the lateral wall 59b may intersect the second end of the lateral wall 60b. So configured, the pairs of lateral walls 59a, 59b, 60a, 60b are separated by an equal distance or a substantially equal distance along the length of each of the first segment 54a and the second segment 54b. The first segment axis 55 may form a first acute angle with the actuator lever axis 38 and the second segment axis 56 may form a second acute angle with the actuator lever axis 38. In some embodiments, the first acute angle and the second acute angle may be identical or substantially identical, and the first acute angle may be between 45° and 80°, or between 60° and 80°. In some embodiments, the actuator lever axis 38 may bisect the cam path 54 such that the actuator lever axis 38 extends between the second end 58b of the first segment 54a and the second end of the second segment 54b such that the first segment 54a and the second segment 54b are symmetrical about the actuator lever axis 38. In some embodiments, the first segment 54a and the second segment 54b may cooperate to form a V-shape, an arc, or a segment of a circle.

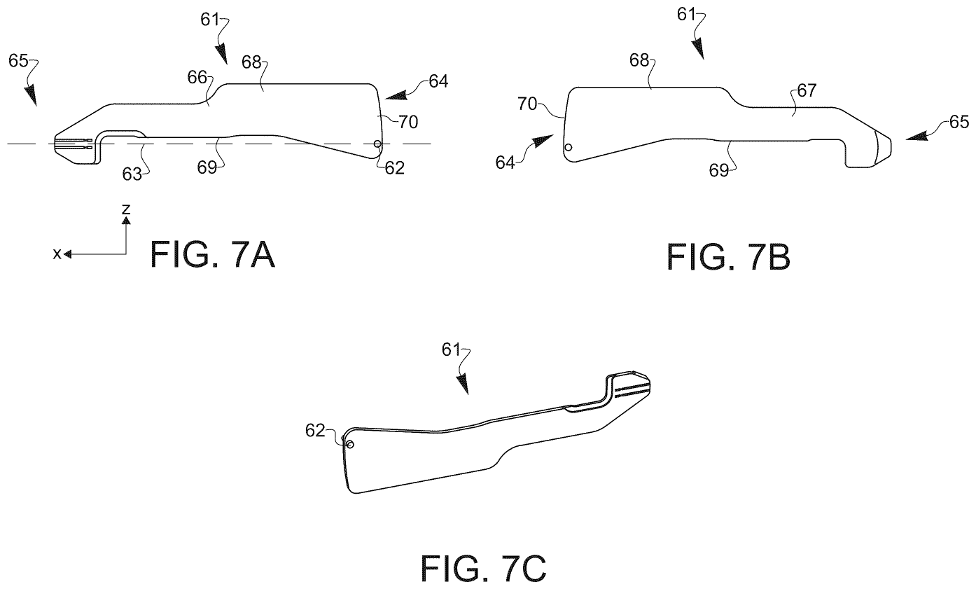

Referring to FIGS. 2A, 5, and 7A to 7C, the suturing device 10 may also include the follower plate 61 that may be coupled to a portion of the housing portion 14 and/or one or more portions of the actuator lever 12. Turning to FIG. 7A, the follower plate 61 may be elongated and may extend along a follower plate axis 63 from a proximal end 64 to a distal end 65, and the follower plate axis 68 may be parallel or substantially parallel to the X-axis of the reference coordinate system of FIG. 7A. The follower plate 61 may be planar and may include a first lateral surface 66 that may extend from the proximal end 64 of follower plate 61 to the distal end 65 of the follower plate 61. As illustrated in FIG. 7B, the follower plate 61 may also include a second lateral surface 67 that may extend from extend from the proximal end 64 of the follower plate 61 to the distal end 65 of the follower plate 61, and the second lateral surface 67 may be offset from the first lateral surface 66 along the Y-axis of the reference coordinate system of FIG. 7A.

The follower plate 61 may be further defined by a top edge portion 68 that may extend in a direction along the follower plate axis 63, and the top edge portion 68 may extend from a first end at or adjacent to the distal end 65 of the follower plate 61 to a second end at or adjacent to the proximal end 64 of the follower plate 61. In some embodiments, the top edge portion 68 may include two or more segments that are not linearly aligned such that the top edge portion 68 is non-linear. The follower plate 61 may also be defined by a bottom edge portion 69 that may extend in a general direction along the follower plate axis 63, and the bottom edge portion 69 may extend from a first end at or adjacent to the distal end 65 of the follower plate 61 to a second end at or adjacent to the proximal end 64 of the follower plate 61. In some embodiments, the bottom edge portion 69 may include two or more segments that are not linearly aligned such that the bottom edge portion 69 is non-linear. In addition, the follower plate 61 may be defined by a proximal end edge 70 that may extend in a general direction normal to the follower plate axis 63, and the proximal end edge 70 may extend from the second end of the top edge portion 68 to the second end of the bottom edge portion 69. In some embodiments, the proximal end edge 70 may include two or more segments, or may include a single segment, and this segment may have a linear or substantially linear shape.

As illustrated in FIGS. 7A and 7C, the cam follower 62 may may extent from a portion of the first lateral surface 66 of the follower plate 61, and the cam follower 62 may be disposed at or adjacent to the proximal end 64 of the follower plate 61, at or adjacent to the second end of the bottom edge portion 69 (e.g., at or adjacent to the intersection of the second end of the bottom edge portion 69 and an end portion of the proximal end edge 70). The cam follower 62 may be cylindrical and may extend along an axis that is parallel to the Y-axis of the reference coordinate system of FIG. 7A. The diameter of the cylinder may be slightly less than the width between the pair of lateral walls 59a, 59b of the first segment 54a of the cam path 54 and the pair of lateral walls 60a, 60b of the second segment 54b of the cam path 54 such that the cylinder may slide along the camp path 54 as will be described in more detail below.

Referring to FIG. 1A, the suturing device 10 also includes a shaft 20 that extends from a proximal end 73 to a distal end 96a along a shaft axis 75, and the shaft axis 75 may be linear and may be parallel or substantially parallel to the X-axis of the reference coordinate system of FIG. 1A. In some embodiments, the shaft axis 75 may be parallel or coaxially-aligned with the housing axis 22. In some embodiments, a portion of the proximal end 73 of the shaft may be disposed at a portion of the housing portion 14, such as a portion of the housing portion 14 that is at or adjacent to the distal end 24 of the housing portion 14. In some embodiments, and all or a portion of the proximal end 73 of the shaft may be integrally formed with a portion of the housing portion 14 at or adjacent to the distal end 24 of the housing portion 14. The shaft 20 may be rigid, but in other embodiments, the shaft 20 may be flexible or may have one or more portions that are flexible.

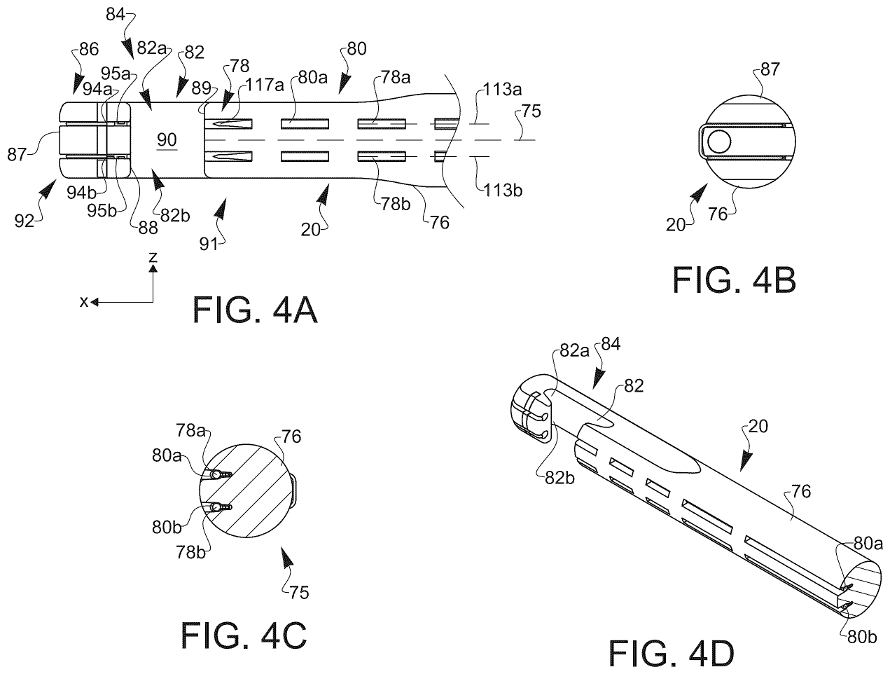

The shaft 20, or one or more portions of the shaft 20, may have the general shape of an elongated hollow tube having one or more interior surfaces that may define an interior portion of the shaft 20 that may extend from the proximal end 73 to the distal end 96a of the shaft 20, The shaft 20 and the one or more interior surfaces may have any suitable cross-sectional shape or combination of shapes normal to the shaft axis 75. For example, the shaft 20 may have the general shape of an elongated cylinder, and the one or more interior surfaces may have a circular cross-sectional shape when viewed normal to the shaft axis 75. In some embodiments, the diameter of the shaft 20 may be 5 mm, which enables fast, precise, and convenient intracorporeal placement in a treatment area. In the embodiment of FIG. 1A, and as illustrated in the cross-sectional view of the shaft 20 of FIGS. 4C and 4D, the shaft 20 may be defined by an exterior surface 76, and one or more apertures 78 may be disposed within the shaft 20 that may extend along or parallel to the shaft axis 75, and each of the one or more apertures 78 may extend from the proximal end 73 to the distal end 96a of the shaft 20. Each of the one or more apertures 78 may be sized and configures to receive a needle 80. For example, the shaft 20 may include a first aperture 78a and a second aperture 78b, and the first aperture 78a and the second aperture 78b are configured to receive a portion of a first needle 80a and a portion of a second needle 80b,

With reference to FIG. 5, the suturing device 10 includes a first needle 80a extending along a first needle axis 113a from a proximal end 115a to a distal end 117a, and the first needle axis 113a may be linear and may be parallel or coaxially aligned with the shaft axis 75. In some embodiments, at least a portion of the first needle 80a extends through or within the first aperture 78a of the shaft 20, as illustrated in FIGS. 4C and 4D. In other embodiments, at least a portion of the first needle 80a extends through the interior portion of the shaft 20. In some embodiments, the proximal end 115a may extend into the interior of the housing portion 14. For example, as illustrated in FIG. 5, the proximal end 115a may be coupled to a first portion of the follower plate 61, such as a first portion of the follower plate at or adjacent to the distal end 65 of the follower plate 61. The first portion may be disposed at or adjacent to the first end of the top edge portion 68 of the follower plate 61 and at or adjacent to the first end of the bottom edge portion 69 of the follower plate 61. A first needle tip 81a is disposed at the distal end 117a of the first needle 80a, and the first needle tip 81a may converge to a sharp point such that the distal end 117a is configured and shaped to pierce tissue that may be disposed in a first portion 82a of a tissue bite area 82 defined in a distal end assembly 84 when the first needle 80a extends from a first needle position (i.e., a retracted position illustrated in FIG. 3A) to a second needle position (i.e., an extended position illustrated in FIG. 3C), as will be described in more detail in a following section.

Turning to FIG. 5, the suturing device 10 also includes a second needle 80b that may be identical or substantially identical to the first needle 80a. In particular, the second needle 80b may extend along a second needle axis 113b from a proximal end 115b to a distal end 117b, and the second needle axis 113b may be linear and may be parallel or coaxially aligned with the shaft axis 75, and the first needle axis 113a may be parallel to and offset from the second needle axis 113b along the Z-axis of the reference coordinate system of FIG. 5. In some embodiments, at least a portion of the second needle 80b extends through or within the second aperture 78b of the shaft 20, as illustrated in FIGS. 4C and 4D. In other embodiments, at least a portion of the second needle 80b extends through the interior portion of the shaft 20. In some embodiments, the proximal end 115b may extend into the interior of the housing portion 14. For example, as illustrated in FIG. 5, the proximal end 115b may be coupled to a second portion of the follower plate 61, such as a second portion of the follower plate at or adjacent to the distal end 65 of the follower plate 61. The second portion may be disposed at or adjacent to the first end of the top edge portion 68 of the follower plate 61 and at or adjacent to the first end of the bottom edge portion 69 of the follower plate 61. A second needle tip 81b is disposed at the distal end 117b of the second needle 80b, and the second needle tip 81b may converge to a sharp point such that the distal end 117b is configured and shaped to pierce tissue that may be disposed in a second portion 82b of the tissue bite area 82 defined in the distal end assembly 84 when the second needle 31b extends from a first needle position (i.e., a retracted position illustrated in FIG. 3A) to a second needle position (i.e., an extended position illustrated in FIG. 3C), as will be described in more detail in a following section.

With reference to FIG. 1A, the suturing device 10 may include the distal end assembly 84 that is formed at or coupled to the distal end 96a of the shaft 20 or a point adjacent to the distal end 96a of the shaft 20. The distal end assembly 84 may extend along or generally along the shaft axis 75, which may be parallel to the X-axis of the reference coordinate system of FIG. 4A, from a proximal end 91 of the distal end assembly 84 to a distal end 92 of the distal end assembly 84, and the proximal end 87 of the distal end assembly 84 may be at or adjacent to the distal end 96a of the shaft 20.

Referring to FIG. 4A, the distal end assembly 84 may be defined by a distal transverse wall 88, a proximal transverse wall 89, and a longitudinal wall 90. The distal transverse wall 88 may be planar or substantially planar, and may extend normal or substantially normal to the shaft axis 75. The distal transverse wall 88 may be proximally offset from the distal end 92 of the distal end assembly 84 along the shaft axis 75 such that a distal base portion 86 is defined between the distal end 92 of the distal end assembly 84 and the distal transverse wall 88. In particular, the distal end 92 of the distal end assembly 84 may be defined by one or more distal end surfaces 87, as illustrated in FIGS. 4A and 4B, and the distal base portion 86 may be defined between the one or more distal end surfaces 87 of the distal end 92 of the distal end assembly 84 and the distal transverse wall 88.

As illustrated in FIG. 4A, a first ferrule recess 94a may be formed in a first portion of the distal base portion 86. In particular, the first ferrule recess 94a may be an aperture that extends through the first portion of the distal base portion 86, and an axis of the first ferrule recess 94a may be aligned with the first needle axis 113a and an axis of the first aperture 78a. The first ferrule recess 94a may extend entirely through the first portion of the distal base portion 86 such that a proximal end of the first ferrule recess 94a is disposed on a first portion of the distal transverse wall 88 and a distal end of the first ferrule recess 94a is disposed on a first portion of the one or more distal end surfaces 87 of the distal end 92 of the distal end assembly 84. A first suture channel 96a may extend from the proximal end of the first ferrule recess 94a to the distal end the first ferrule recess 94a to allow suture coupled to the distal end of a first ferrule 95a to be inserted into and removed from the first suture channel 96a. A proximal end of the first ferrule 95a may be disposed at or adjacent to the proximal end of the first ferrule recess 94a, and a distal end of the first ferrule 95a may be proximal to the one or more distal end surfaces 87 of the distal end 92. The first ferrule recess 94a may be sized and configured to retain the first ferrule 95a when the first needle tip 81a of the first needle 80a is in the first needle position, but is configured to allow the first ferrule 95a to operatively couple to the first needle tip 81a of the first needle 80a when the first needle 80a displaces from the second needle position to the first needle position, thereby pulling the first ferrule 95a (and the suture coupled to the first ferrule 95a) through an aperture formed in the tissue disposed within the first portion 82a of tissue bite area 82. One or more locking or release mechanisms may be disposed at or adjacent to (and/or remote from) the first ferrule recess 94a to selectively retain and release the first ferrule 95a.

In addition, a second ferrule recess 94b may be formed in a second portion of the distal base portion 86, and the second ferrule recess 94b may be identical to the first ferrule recess 94a may be formed in the first portion of the distal base portion 86. In particular, the second ferrule recess 94b may be an aperture that extends through the second portion of the distal base portion 86, and an axis of the second ferrule recess 94b may be aligned with the second needle axis 113b and an axis of the second aperture 78b. The second ferrule recess 94b may extend entirely through the second portion of the distal base portion 86 such that a proximal end of the second ferrule recess 94b is disposed on a second portion of the distal transverse wall 88 and a distal end of the second ferrule recess 94b is disposed on a second portion of the one or more distal end surfaces 87 of the distal end 92 of the distal end assembly 84. A second suture channel 96b may extend from the proximal end of the second ferrule recess 94b to the distal end the second ferrule recess 94b to allow suture coupled to the distal end of a second ferrule 95b to be inserted into and removed from the second suture channel 96b. A proximal end of the second ferrule 95b may be disposed at or adjacent to the proximal end of the second ferrule recess 94b, and a distal end of the second ferrule 95b may be proximal to the one or more distal end surfaces 87 of the distal end 92. The second ferrule recess 94b may be sized and configured to retain the second ferrule 95b when the second needle tip 81b of the second needle 80b is in the first needle position, but is configured to allow the second ferrule 95b to operatively couple to the second needle tip 81b of the second needle 80b when the second needle 80a displaces from the second needle position to the first needle position, thereby pulling the second ferrule 95b (and the suture coupled to the second ferrule 95b) through an aperture formed in the tissue disposed within the second portion 82b of tissue bite area 82. One or more locking or release mechanisms may be disposed at or adjacent to (and/or remote from) the second ferrule recess 94b to selectively retain and release the second ferrule 95b.

Each of the first and second ferrules 95a, 95b may be any ferrule that is configured to be secured to a portion of suture extending from the distal end of the first and second ferrules 95a, 95b and that is configured to be selectively coupled to and uncoupled from the first and second needle tips 81a, 81b, respectively, and the first and second ferrules 95a, 95b may be similar to the ferrule disclosed in U.S. Pat. No. 10,390,818, which issued on Aug. 27, 2019 and which is herein incorporated by reference in its entirety.

As illustrated in FIG. 4A, the proximal transverse wall 89 may be planar or substantially planar, and may extend normal or substantially normal to the shaft axis 75. The proximal transverse wall 89 may be proximally offset from the distal transverse wall 88 along the shaft axis 75, and the proximal transverse wall 89 may be disposed at or adjacent to the proximal end 91 of the distal end assembly 84. A distal end of each of the first aperture 78a and the second aperture 78b may extend through the proximal transverse wall 89. The longitudinal wall 90 may be planar or substantially planar, and may extend between the distal transverse wall 88 and the proximal transverse wall 89 along or substantially along the shaft axis 75. One or more portions of the distal transverse wall 88, the proximal transverse wall 89, and the longitudinal wall 90 may cooperate to define the tissue bite area 82, which may be a slot formed through a portion of the distal end assembly 84, and the slot may extend through a portion of the distal end assembly 84 from the distal transverse wall 88 to the proximal transverse wall 89 along the shaft axis 75.

When the first needle 80a is in the first needle position (i.e., the retracted position illustrated in FIG. 3A), the first needle tip 81a of the first needle 80a is disposed proximal to the proximal transverse wall 89 such that no portion of the first needle tip 81a of the first needle 80a is disposed or extends into the first portion 82a of a tissue bite area 82. Also in the first needle position, the second needle tip 81b of the second needle 80b is disposed proximal to the proximal transverse wall 89 such that no portion of the second needle tip 81b of the second needle 80b is disposed or extends into the second portion 82b of a tissue bite area 82

Turning to FIG. 3B, when the first needle tip 81a of the first needle 80a begins to displace distally from the first needle position towards the second needle position (i.e., the extended position illustrated in FIG. 3C), the first needle tip 81a of the first needle 80a extends through the distal end of the first aperture 78a and into the first portion 82a of a tissue bite area 82. In addition, when the second needle tip 81b of the second needle 80b begins to displace distally from the first needle position towards the second needle position (i.e., the extended position illustrated in FIG. 3C), the second needle tip 81b of the second needle 80b extends through the distal end of the second aperture 78b and into the second portion 82b of a tissue bite area 82.

As illustrated in FIG. 3C, the first needle tip 81a of the first needle 80a fully displaces distally through the first portion 82a of a tissue bite area 82 and into the second needle position in which the first needle tip 81a extends into the first ferrule recess 94a and operatively engages the first ferrule 95a disposed in the first ferrule recess 94a. In addition, the second needle tip 81b of the second needle 80b fully displaces distally through the second portion 82b of the tissue bite area 82 and into the second needle position in which the second needle tip 81b of the second needle 80b extends into the second ferrule recess 94b and operatively engages the second ferrule 95b disposed in the second ferrule recess 94b.

Turning to FIG. 3D, when the first needle tip 81a of the first needle 80a begins to displace proximally from the second needle position towards the first needle position, the first needle tip 81a of the first needle 80a, and the first ferrule 95a coupled to the first needle tip 81a, extends through the first portion 82a of a tissue bite area 82 and towards the proximal end 91 of the distal end assembly 84. In addition, when the second needle tip 81b of the second needle 80b begins to displace proximally from the second needle position towards the first needle position, the second needle tip 81b of the second needle 80b, and the second ferrule 95b coupled to the second needle tip 81b, extends through the second portion 82b of a tissue bite area 82 and towards the proximal end 91 of the distal end assembly 84.

With reference to FIG. 3E, the first needle tip 81a of the first needle 80a fully displaces proximally through the first portion 82a of a tissue bite area 82 and into the first needle position in which the first needle tip 81a of the first needle 80a is disposed proximal to the proximal transverse wall 89. In some embodiments, the proximal end of the first ferrule 95a is disposed proximal to the proximal transverse wall 89, and in other embodiments, both the proximal end and the distal end of the first ferrule 95a are disposed proximal to the proximal transverse wall 89. In addition, the second needle tip 81b of the second needle 80b fully displaces proximally through the second portion 82b of a tissue bite area 82 and into the first needle position in which the second needle tip 81b of the second needle 80b is disposed proximal to the proximal transverse wall 89. In some embodiments, the proximal end of the second ferrule 95b is disposed proximal to the proximal transverse wall 89, and in other embodiments, both the proximal end and the distal end of the second ferrule 95b are disposed proximal to the proximal transverse wall 89.

As previously explained, the proximal end 115a of the first needle 80a may be coupled to the first portion of the follower plate 61 and the proximal end 115b of the second needle 80b may be coupled to the second portion of the follower plate 61, and the cam follower 62 may be disposed in the cam path 54 of the actuator lever 12. With reference to FIG. 2A, when the actuation lever 12 is in the unactuated first lever position, the cam follower 62 may be disposed at or adjacent to the first end 58a of the first segment 54a (as illustrated in FIG. 6A) and the follower plate 61 is disposed in an unactuated proximal position. In this position, the first needle 80a and the second needle 80b are in the first needle position (illustrated in FIG. 3A). In use, a surgeon may grasp the suturing device 10 around a portion of the housing portion 14 and a portion of the actuation lever 12 such that two or more fingers are positioned on or along the bottom edge portion 46 of the actuation lever 12. In addition, the surgeon may position tissue to be sutured in the tissue bite area 82 of the distal end assembly 84.

The surgeon may begin the suturing process by applying an upward force with the fingers against the bottom edge portion 46 of the actuation lever 12. This upward force causes the actuation lever 12 to begin to be displaced from the unactuated first lever position towards the intermediate second lever position (illustrated in FIG. 2B) by rotating the actuation lever 12 about the rotational axis 44 (illustrated in FIG. 6C) in a first rotational direction, and the rotation of the actuation lever 12 (and, in particular, the rotation of the cam path 54 of the actuation lever 12) relative to the follower plate 61 causes the cam follower 62 to displace from the first end 58a of the first segment 54a (as illustrated in FIG. 6A) towards the second end 58b of the first segment 54a. Because of the declined angle of the first segment 54a of the cam path 54, the follower plate 61 is displaced distally (along or substantially along the flower plate axis 63) within the interior portion 35 of the housing portion 14 from the unactuated proximal position towards an actuated distal position. As the follower plate 61 displaces distally, the first needle 80a and the second needle 80b displace from the first needle position towards the second needle position (as illustrated in FIG. 3B). Accordingly, the first needle tip 81a begins to pierce a first portion of the tissue disposed within the first portion 82a of the tissue bite area 82 and the second needle tip 81b begins to pierce a second portion of the tissue disposed within the second portion 82b of the tissue bite area 82.

One or more interior surfaces that define the interior portion 35 of the housing portion 14 may interact with one or more edges of the follower plate 61 to guide the follower plate 61 as it moves between the unactuated proximal position (illustrated in FIGS. 2A and 2C) and the actuated distal position (illustrated in FIG. 2B) along (or substantially along) the follower plate axis 63. For example, one or more interior surfaces or edges corresponding to one or more portions of the top edge portion 28 of the housing portion 14 (see FIG. 2A) may contact one or more portions of the top edge portion 68 of the follower plate 61 to guide the follower plate 61. Additionally, or alternatively, one or more interior surfaces or edges corresponding to one or more portions of the bottom edge portion 30 of the housing portion 14 may contact one or more portions of the bottom edge portion 69 of the follower plate 61 to guide the follower plate 61.

As the actuation lever 12 continues to be rotated about the rotational axis 44 in the first rotational direction, the actuation lever 12 is displaced to the intermediate second lever position (illustrated in FIG. 2B). In the intermediate second lever position, the rotation of the actuation lever 12 and the cam path 54 relative to the follower plate 61 causes the cam follower 62 to displace to the second end 58b of the first segment 54a and/or to the second end 57b of the second segment 54b (illustrated in FIG. 6A), where the follower plate 61 is in the actuated distal position. In the actuated distal position of the follower plate 61, the first needle 80a and the second needle 80b are displaced into the second needle position (as illustrated in FIG. 3B), and the first needle tip 81a and the second needle tip 81b operatively engage the first ferrule 95a and the second ferrule 95b, respectively.

As the actuation lever 12 is continued to be displaced by the surgeon from the intermediate second lever position towards the fully actuated third lever position (illustrated in FIG. 2C) by rotating the actuation lever 12 about the rotational axis 44 in the first rotational direction, the rotation of the actuation lever 12 and the cam path 54 relative to the follower plate 61 causes the cam follower 62 to displace from the second end 57b of the second segment 54b towards the first end 57a of the second segment 54b. Because of the inclined angle of the second segment 54b of the cam path 54, the follower plate 61 reverses direction along the flower plate axis 63 and is displaced proximally (along or substantially along the flower plate axis 63) within the interior portion 35 of the housing portion 14 from the actuated distal position towards the unactuated proximal position. As the follower plate 61 begins to displace towards the unactuated proximal position, the first needle 80a and the second needle 80b begin to displace from the second needle position towards the first needle position (as illustrated in FIG. 3D). Accordingly, the first needle tip 81a displaces the attached first ferrule 95a through the channel or path formed in the first portion of the tissue disposed within the first portion 82a of the tissue bite area 82 and the second needle tip 81b displaces the attached second ferrule 95b through the channel or path formed in the second portion of the tissue disposed within the second portion 82b of the tissue bite area 82. This displacement of the first ferrule 95a and the second ferrule 95b displaces the suture through the channel or path formed in the first and second portions of the tissue, respectively.

When the actuation lever 12 is displaced into the fully actuated third lever position, the rotation of the actuation lever 12 and the cam path 54 relative to the follower plate 61 causes the cam follower 62 to displace to the first end 57a of the second segment 54b such that the follower plate 61 is in the unactuated proximal position. With the follower plate 61 in the unactuated proximal position, the first needle 80a and the second needle 80b are displaced into the first needle position (as illustrated in FIG. 3E) with the first ferrule 95a and the second ferrule 95b coupled to the first needle tip 81a and the second needle tip 81b, respectively. Thus, the suture coupled to the distal ends of the first ferrule 95a and the second ferrule 95b extends completely through the channel or path formed in the first and second portions of the tissue, respectively. If a single length of suture is coupled to the distal ends of the first ferrule 95a and the second ferrule 95b, the first ferrule 95a and the second ferrule 95b may be removed from the first needle tip 81a and the second needle tip 81b, respectively, and the free ends of the suture may extend from the first portion of the tissue and the second portion of the tissue.

In some embodiments, the first ferrule 95a and the second ferrule 95b may be stripped from the first needle tip 81a and the second needle tip 81b, respectively, by one or more corresponding features disposed at or adjacent to the distal ends of each of the first and second apertures 78a, 78b extending through the shaft 20.

As previously explained, with the actuation lever 12 is displaced into the fully actuated third lever position, the lock feature 52 of the actuation lever 12 may engage the one or more of the plurality of ratchet features 53 formed along an inner profile of the proximal end edge 32 of the housing portion 14, as illustrated in FIG. 8, thereby maintaining the actuation lever 12 in the fully actuated third lever position. This automatic locking mechanism prevents further unintended actuation of the first and second needles 80a, 80b after use, allowing the suturing device 10 to be safely discarded. In some embodiments, all or portions of the housing portion 14, the actuation lever 12, the follower plate 61, the shaft 20, and the distal end assembly 84 may be made of plastic (e.g., fabricated as injection molded plastic parts) such that the suturing device 10 is intended to be disposable and not reusable, while reducing manufacturing cost and complexity.

While the disclosed embodiment of the suturing device 10 includes both the first needle 80a and the second needle 80b, some embodiments may only include the first needle 80a, and such embodiments may have only a first ferrule recess 94a that secures a first ferrule 95a. In other embodiments, the suturing device 10 may include the first needle 80a, the second needle 80b, and a third needle (not shown) that may operate identically to the first needle 80a and the second needle 80b to engage a third ferrule (not shown) disposed in a third ferrule recess (not shown). A fourth needle (not shown) may also be included.

Various advantages of the suturing device 10 have been discussed above. Embodiments discussed herein have been described by way of example in this specification. It will be apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. The drawings included herein are not necessarily drawn to scale. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A suturing device comprising:
a housing having one or more surfaces defining an interior portion;
an actuation lever coupled to a first portion of the housing, the actuation lever having a grip portion that is configured to be engaged by a user, wherein a slot defining a cam path is formed in a first portion of the actuation lever, the actuation lever configured to be displaceable from an unactuated first lever position to an intermediate second lever position to a fully actuated third lever position by displacing the actuation lever in a single direction;
a follower plate that includes a cam follower that is disposed within the slot defining the cam path of the actuation lever such that (a) as the actuation lever is displaced from the unactuated first lever position to the intermediate second lever position, the follower plate is displaced from an unactuated proximal position to an actuated distal position, and (b) as the actuation lever is displaced from the intermediate second lever position to the fully actuated third lever position, the follower plate is displaced from the actuated distal position to the unactuated proximal position; and
a first needle that extends from a proximal end to a distal end along a first needle axis, with a first needle tip being disposed at the distal end of the first needle, wherein the proximal end of the first needle is coupled to a first portion of the follower plate such that (a) as the follower plate is displaced from the unactuated proximal position to the actuated distal position, the first needle is displaced from a first needle position to a second needle position, and such that (b) as the follower plate is displaced from the actuated distal position to the unactuated proximal position to the actuated distal position, the first needle is displaced from the second needle position to the first needle position.

2. The suturing device of claim 1, wherein the cam path includes a first segment and a second segment.

3. The suturing device of claim 2, wherein the first segment is an elongated first slot that extends from a first end to a second end along a first segment axis, and the second segment is an elongated second slot that extends from a first end to a second end along a second segment axis.

4. The suturing device of claim 3, wherein each of the first segment axis and the second segment axis is linear.

5. The suturing device of claim 4, wherein the first segment axis and the second segment axis form a V-shape.

6. The suturing device of claim 1, further comprising a shaft coupled to a second portion of the housing, wherein the shaft extends from a proximal end to a distal end along a shaft axis.

7. The suturing device of claim 6, wherein a portion of the first needle extends through the interior portion of the shaft.

8. The suturing device of claim 6, further comprising a distal end assembly disposed at the distal end of the shaft, wherein one or more surfaces of the distal end assembly define a first tissue bite area such that as the first needle translates from the first needle position to the second needle position, the first needle tip is configured to pierce a first portion of tissue disposed within a first portion of the tissue bite area.

9. The suturing device of claim 6, wherein a first ferrule recess is formed in a first portion of the distal end assembly that is distal to the tissue bite area such that when the first needle is in the second needle position, the first needle tip is configured to engage a first ferrule that is configured to be disposed within a portion of the first ferrule recess, wherein the first ferrule is configured to be coupled to a portion of suture, and
wherein the first ferrule is configured to be secured to the first needle tip as the first needle translates from the first needle position to the second needle position.

10. The suturing device of claim 1, further comprising a cantilevered arm coupled to a second portion of the actuation lever, the cantilevered arm having a lock feature at end of the distal arm, the lock feature configured to engage one or more of a plurality of ratchet features formed on a third portion of the housing when the actuation lever is in the fully actuated third lever position to maintain the actuation lever in the fully actuated third lever position.

11. The suturing device of claim 1, wherein the follower plate is elongated and extends from a proximal end to a distal end, and the cam follower is disposed at or adjacent to the proximal end of the follower plate.

12. The suturing device of claim 11, wherein the proximal end of the first needle is coupled to the first portion of the follower plate which is disposed at the distal end of the follower plate.

13. The suturing device of claim 1, wherein the follower plate is disposed within the interior portion of the housing.

14. The suturing device of claim 1, further comprising:
a second needle that extends from a proximal end to a distal end along a second needle axis, with a second needle tip being disposed at the distal end of the second needle, wherein the proximal end of the second needle is coupled to a second portion of the follower plate such that (a) as the follower plate is displaced from the unactuated proximal position to the actuated distal position, the second needle is displaced from a first needle position to a second needle position, and (b) as the follower plate is displaced from the actuated distal position to the unactuated proximal position to the actuated distal position, the second needle is displaced from the second needle position to the first needle position.

15. The suturing device of claim 14, wherein the first needle axis is parallel to and offset from the second needle axis.

16. The suturing device of claim 3, wherein the cam follower is displaced from the first end of the first slot to the second end of the first slot as the follower plate is displaced from the unactuated proximal position to the actuated distal position, and wherein the cam follower is displaced from the first end of the second slot to the second end of the second slot as the follower plate is displaced from the actuated distal position to the unactuated proximal position, and wherein the second end of the first slot is disposed at or adjacent to the first end of the second slot.

17. The suturing device of claim 1, wherein the grip portion of the actuation lever is disposed external to the housing when the actuation lever is in the unactuated first lever position and the intermediate second lever position.

18. The suturing device of claim 1, wherein the housing, the actuation lever, and the follower plate are each injection-molded plastic parts.

19. The suturing device of claim 1, wherein the follower plate is planar and includes a planar first surface and a planar second surface, and the cam follower is a cylindrical protrusion that extends from the first surface.

20. A single-use suturing device comprising:
a housing having one or more surfaces defining an interior portion;
a shaft coupled to a first portion of the housing, wherein the shaft extends from a proximal end to a distal end along a shaft axis
an actuation lever pivotably coupled to a second portion of the housing within the interior portion, the actuation lever having a grip portion that is configured to be engaged by a user, wherein a slot defining a cam path is formed in a first portion of the actuation lever, wherein the cam path includes a first segment and a second segment that cooperate to form a V-shape, the actuation lever configured to be pivotable from an unactuated first lever position to an second lever position to a fully actuated third lever position by displacing the actuation lever in a single direction, and the actuation lever having a grip portion that is disposed external to the housing when the actuation lever is in the unactuated first lever position and the intermediate second lever position, wherein when the actuation lever is configured to be locked in the fully actuated third lever position when the actuation lever is displaced to the fully actuated third lever position;
a follower plate that includes a cam follower that is disposed within the slot defining the cam path of the actuation lever such that (a) as the actuation lever is displaced from the unactuated first lever position to the intermediate second lever position, the follower plate is displaced from an unactuated proximal position to an actuated distal position, and (b) as the actuation lever is displaced from the intermediate second lever position to the fully actuated third lever position, the follower plate is displaced from the actuated distal position to the unactuated proximal position, wherein the cam follower is displaced from the first end of the first slot to the second end of the first slot as the follower plate is displaced from the unactuated proximal position to the actuated distal position, and wherein the cam follower is displaced from the first end of the second slot to the second end of the second slot as the follower plate is displaced from the actuated distal position to the unactuated proximal position, and wherein the second end of the first slot is disposed at or adjacent to the first end of the second slot, and wherein the follower plate is disposed within the interior portion of the housing;
a first needle that extends from a proximal end to a distal end along a first needle axis, with a first needle tip being disposed at the distal end of the first needle, wherein the proximal end of the first needle is coupled to a first portion of the follower plate such that (a) as the follower plate is displaced from the unactuated proximal position to the actuated distal position, the first needle is displaced from a first needle position to a second needle position, and such that (b) as the follower plate is displaced from the actuated distal position to the unactuated proximal position to the actuated distal position, the first needle is displaced from the second needle position to the first needle position;
a second needle that extends from a proximal end to a distal end along a second needle axis, with a second needle tip being disposed at the distal end of the second needle, wherein the proximal end of the second needle is coupled to a second portion of the follower plate such that (a) as the follower plate is displaced from the unactuated proximal position to the actuated distal position, the second needle is displaced from a first needle position to a second needle position, and (b) as the follower plate is displaced from the actuated distal position to the unactuated proximal position to the actuated distal position, the second needle is displaced from the second needle position to the first needle position, wherein the first needle axis is parallel to and offset from the second needle axis; and
a distal end assembly disposed at the distal end of the shaft, wherein one or more surfaces of the distal end assembly define a first tissue bite area such that (a) as the first needle translates from the first needle position to the second needle position, the first needle tip is configured to pierce a first portion of tissue disposed within a first portion of the tissue bite area, and (b) as the second needle translates from the first needle position to the second needle position, the second needle tip is configured to pierce a second portion of the tissue disposed within a second portion of the tissue bite area.
